(19) United States
(12) Patent Application Publication
(10) Pub. No.: US 2026/0123856 A1
(43) Pub. Date: May 7, 2026

(54) EXTRAORAL HOLDER FOR INTRAORAL MEASUREMENTS

(71) Applicant: Ivoclar Vivadent AG, Schaan (LI)

(72) Inventors: Alexander SKAWRAN, Sargans (CH); Marvin KUHN, Feldkirch (AT); Alexander GROSSE-HONEBRINK, Oberriet (CH)

(21) Appl. No.: 19/497,445

(22) PCT Filed: Jul. 6, 2023
(86) PCT No.: PCT/EP2023/068730

§ 371 Date: Dec. 26, 2025

(51) Int. Cl. A61B 5/145; A61B 5/00; A61B 10/00

(52) U.S. Cl. CPC A61B 5/14507; A61B 5/6803; A61B 10/0051

(57) ABSTRACT

The present invention relates to a body-worn device (100) for a dental sensor, comprising a holding element (103) for holding the dental sensor in or on the mouth of a person (107); and a carrier element (105) for supporting the holding element (103) on the body of the person (107).

The present invention relates to a body-worn device for a dental sensor and a method for wearing a dental sensor.

Lactate measurements are performed by blood tests, during which blood is taken at specified intervals at different performance levels. The lactate values are then determined for the various blood samples. Only trained personnel may take the blood samples. The blood samples can be contaminated by sweat, which can distort the measured values. This conventional method is painful and only allows for selective measurements. However, measurements in saliva allow for continuous and painless measurement of lactate values, which can increase the significance of lactate measurements for athletes.

In previous measurements of parameters such as lactate in saliva, only random samples have been taken. This method is impractical for short measurements such as a lactate test, which typically lasts only about half an hour and is usually performed only two to three times a year.

It is the technical task of the present invention to perform continuous measurement in or on a person's oral cavity.

This task is solved by subject-matter according to the independent claims. Technically advantageous embodiments are the subject-matter of the dependent claims, the description, and the drawings.

According to a first aspect, the technical task is solved by a body-worn device for a dental sensor, comprising a holding element for holding the dental sensor in or on the mouth of a person; and a carrier element for supporting the holding element on the body of the person. This body-worn device can be used, for example, to enable continuous measurements in saliva. The body-worn device allows values in saliva or in the intraoral area to be continuously monitored over a period of a few minutes to hours.

In a technically advantageous embodiment of the body-worn device, it comprises a dental sensor. This provides the technical advantage that dental measurements, such as the measurement of lactate values in saliva, can be carried out in a simple manner.

In a further technically advantageous embodiment of the body-worn device, the dental sensor is arranged at the tip of the holding element. This offers the technical advantage that the dental sensor can be easily inserted into the oral cavity and is comfortable to wear there.

In a further technically advantageous embodiment of the body-worn device, the dental sensor is replaceably attached to the holding element. This provides the technical advantage that the dental sensor can be easily replaced for cleaning. The dental sensor can thus be replaced for selective measurements by plugging it in and out. Therefore, it does not need to be worn all the time, for example, or can be replaced with another dental sensor between individual measurement points. Different parameters can also be recorded one after the other, such as a lactate value first and then a glucose value.

In a further technically advantageous embodiment of the body-worn device, the dental sensor comprises an electrochemical or enzymatic dental sensor or a pH sensor, a lactate sensor, a glucose sensor, an alcohol sensor, an O2 sensor, a CO2 sensor, a CO sensor, a VO2Max sensor, a respiratory rate sensor, a noise sensor, a temperature sensor, a pressure sensor, a conductivity sensor, a humidity sensor, a potassium sensor, a sodium sensor, a chloride sensor, a cortisol sensor, a calcium sensor, a sulfate sensor, a microphone, and/or a VOC (volatile organic compounds) sensor. This provides the technical advantage that suitable parameters can be measured sequentially or simultaneously.

In a further technically advantageous embodiment of the body-worn device, the holding element is formed by a wire or a plastic rod. This provides the technical advantage that the holding element can be easily adapted to the individual conditions of a user.

In a further technically advantageous embodiment of the body-worn device, the holding element is flexible. This provides the technical advantage that the holding element can be easily adapted to the individual conditions of a user.

In a further technically advantageous embodiment of the body-worn device, the holding element comprises a fluid tube for a dental sensor. This provides the technical advantage that the fluid tube can serve as a suction tube to suction saliva during the measurement and ensure that the dental sensor remains wet. The suction device maintains a flow of saliva to the dental sensor 101. It also ensures that the saliva on the sensor surface is replaced.

In a further technically advantageous embodiment of the body-worn device, the fluid tube comprises a replaceable headpiece. This provides the technical advantage that it can be replaced easily and quickly to ensure the necessary hygiene.

In a further technically advantageous embodiment of the body-worn device, the headpiece comprises openings through which saliva can be suctioned. This provides the technical advantage that saliva can be effectively suctioned through these openings.

In a further technically advantageous embodiment of the body-worn device, the carrier element is formed by a headset, glasses, or headgear. This provides the technical advantage that the wearing comfort for the user and the acceptance of the body-worn device are increased.

In a further technically advantageous embodiment of the body-worn device, the holding element and the carrier element are detachably connected to each other. This provides the technical advantage that the individual parts of the body-worn device can be easily replaced.

In a further technically advantageous embodiment of the body-worn device, it comprises an energy storage device for supplying the dental sensor with electrical energy. This provides the technical advantage that an external energy source and external cables for supplying power to the dental sensor are not required. This also enables measurements to be taken while the user is not in the same place, such as during a sprint or while running.

In a further technically advantageous embodiment of the body-worn device, the carrier element can be attached to the head or body of the person. This provides the technical advantage that the body-worn device does not need to be held, so that the user has their hands free during the measurement. This also increases the wearing comfort of the body-worn device.

According to a second aspect, the technical task is solved by a method for wearing a dental sensor. The method according to the invention comprises the steps of holding the dental sensor in or on the mouth of a person by means of a holding element; and supporting the holding element on the body of the person by means of a carrier element. This method achieves the same technical advantages as the body-worn device.

Exemplary embodiments of the invention are shown in the drawings and are described in more detail below, in which:

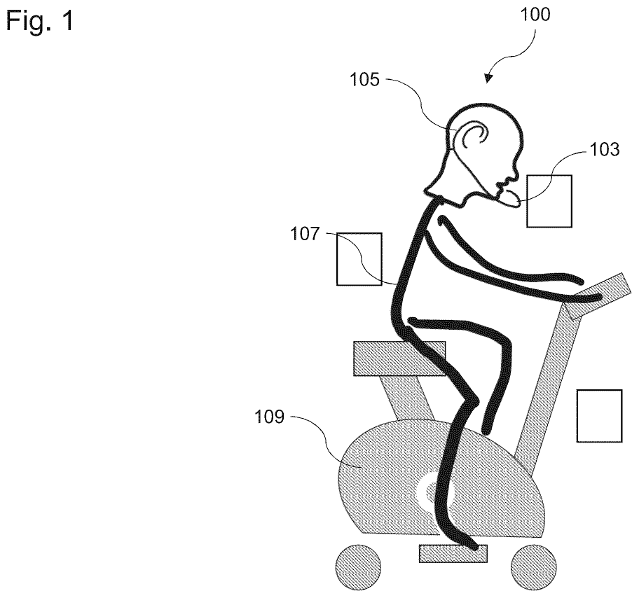

FIG. 1 shows a schematic illustration of a worn body-worn device for a dental sensor;

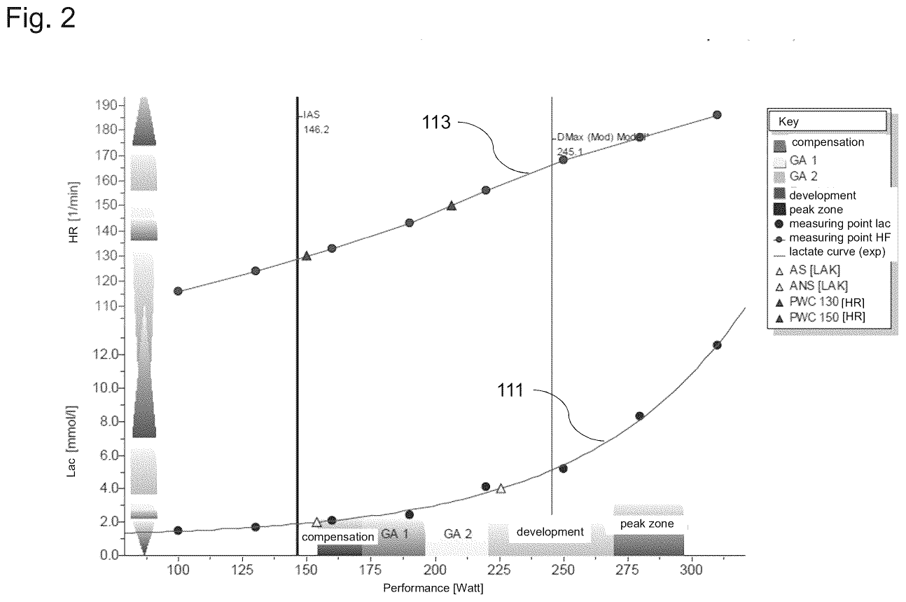

FIG. 2 shows a graphical illustration of exemplary measurement results of a lactate measurement with a body-worn device for a dental sensor;

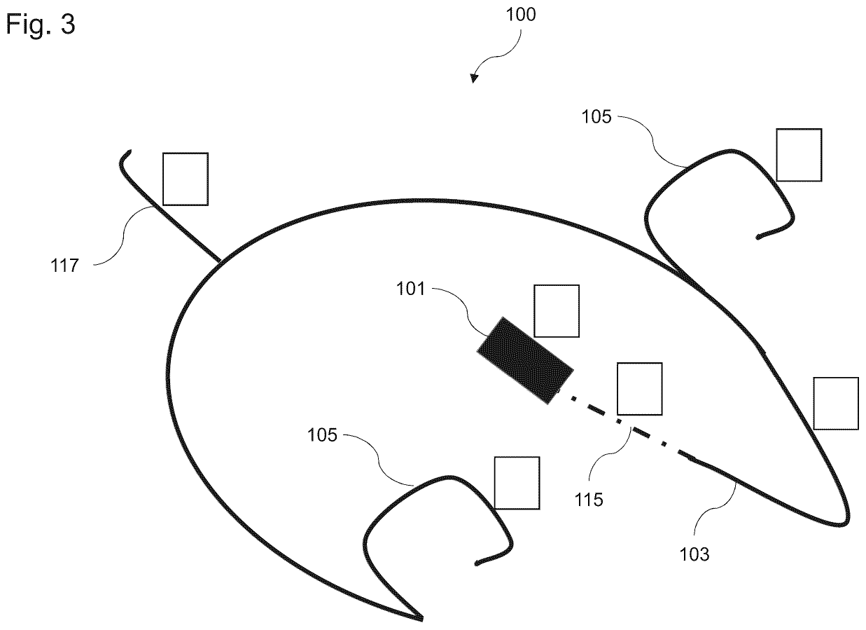

FIG. 3 shows a schematic illustration of a body-worn device for a dental sensor;

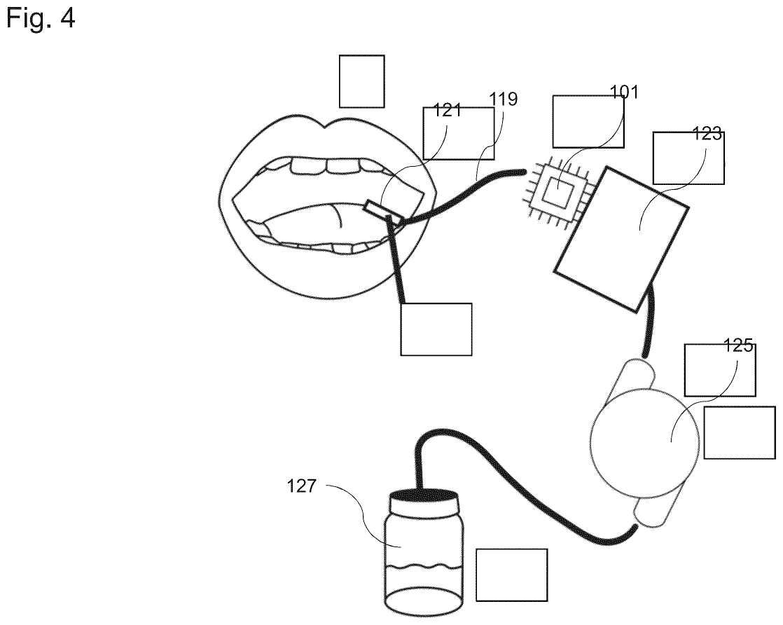

FIG. 4 shows a schematic illustration of a body-worn device for a suction tube; and

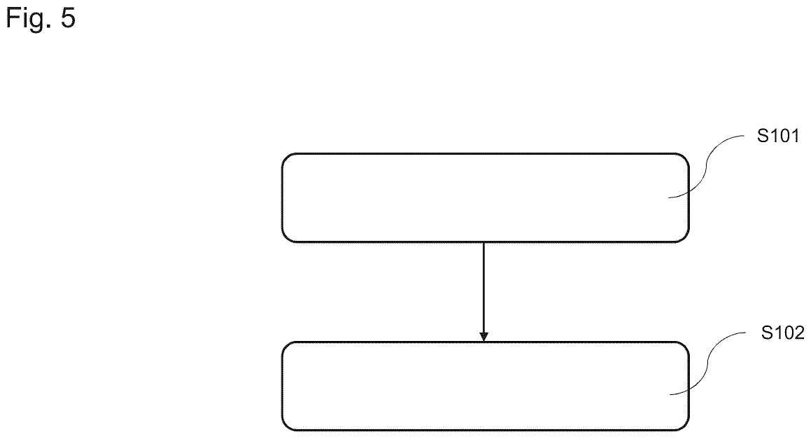

FIG. 5 shows a schematic illustration of a method for wearing a dental sensor.

FIG. 1 shows a schematic illustration of a body-worn device 100 for a dental sensor, which is worn on the head by a user. The body-worn device 100 comprises a holding element 103 for holding a dental sensor in or on the mouth of a person 107 and a carrier element 105 for supporting the holding element 103 on the body of the person 107. The carrier element 105 may be designed as a bracket with which the body-worn device 100 can be attached to the ear of the person 107. In general, the carrier element 105 is any element with which the force of the holding element 103 can be transferred to the body. The holding element 103 is attached to the carrier element 105 and is designed to hold a dental sensor in or on the mouth of a person 107.

The dental sensor, which can be attached to the holding element 103, can be used, for example, for continuous measurements in the saliva of a person 107, such as a lactate test in saliva. When measuring lactate with the body-worn device 100, the person 107 sits, for example, on an ergometer 109 and wears the body-worn device 100 with the carrier element 105 on their head. The holding element 103 is connected to the carrier element 105 and holds the dental sensor in the mouth of the person 107.

The dental sensor can then measure the lactate values of the person 107 depending on the performance of the person 107 on the ergometer 109. Such measurements in saliva using the body-worn device 100 allow continuous and painless measurement of the lactate levels of the person 107. The results can be used to determine current fitness and create an optimal training plan for the person 107.

The dental sensor can be designed to measure other parameters that are important for athletes or other target groups.

Similarly, several dental sensors can be attached to the holding element 103, for example by means of a sensor holder.

If sufficiently small dental sensors are used, it is technically easily realizable to attach one or more dental sensors. Readout electronics for the signals from the dental sensor can be arranged outside the mouth of the person 107. This reduces the space required in the mouth of a user, as the readout electronics and the dental sensor are used separately. The associated readout electronics can be designed to any size and energy consumption. This increases the possible uses of the available dental sensors. The dental sensor and the readout electronics are physically connected via cables. The readout electronics can be carried by a user on a belt or in a pocket.

FIG. 2 shows a graphical illustration of exemplary measurement results from a lactate measurement using the body-worn device 100 for dental sensors. Continuous and painless measurement in saliva allows significant lactate and heart rate values 111 and 113 of the test person to be obtained depending on the performance achieved. This also increases the significance of the lactate measurements for the athlete or the respective trainer.

FIG. 3 shows a schematic illustration of a body-worn device 100 for a dental sensor 101. The extraoral carrier element 105 can be designed in the form of a headset with attachment options to the body, a headband, glasses, or headgear, such as a hat or cap. The carrier element 105 can be made of plastic.

The holding element 103 is formed, for example, by a metal wire or a plastic bracket that is mechanically connected to the carrier element 105. The holding element 103 can be rotatably connected to the carrier element, for example via a ball joint. This allows the body-worn device 100 to be easily adapted to the anatomical conditions of the user.

The holding element 103 has a fixing device 115 for fixing the dental sensor 101 at any position in the mouth. The fixing device 115 may, for example, be a flexible wire on which the dental sensor 101 is arranged and which can be positioned anywhere in the mouth of the person 107. The fixing device 115 is connected to the holding element 103. It is also possible to attach the dental sensor 101 to one or more teeth using a wire or plastic bracket, a temporary adhesive that dissolves after a certain period of time, or suction cups as the fixing device 115.

Since attachment in the mouth of the person 107 is intended for a short period of time, i.e., from a few minutes to a few hours, no individually manufactured carrier surface is required. The dental sensor 101 can also be connected to the carrier element 105 via a holding element 103, which is designed to correspond to a tobacco pipe. There is no risk of swallowing, as the dental sensor 101 and its cabling are connected to the carrier element 105 via the fixing device 115 and the holding element 103.

An interface 117 serves to supply the dental sensor 101 with power and to ensure data exchange with the readout electronics. The cables for this interface 117 can run inside the body-worn device 100 to prevent them from accidentally getting caught. It is also possible for the interface 117 to be wireless. For this purpose, an electrical circuit can be provided that comprises a power source and an interface for wireless data transmission.

FIG. 4 shows a schematic illustration of a body-worn device 100 to which a suction tube 119 is attached. The suction tube 119 can be attached to the body-worn device 100 in place of the dental sensor 101 in order to suction saliva from the mouth. The suction tube 119 may comprise a replaceable head 121 that can be replaced after a measurement to ensure hygiene. The head 121 contains openings through which saliva is suctioned from the mouth. The head 121 of the suction tube 119 may have the same attachment devices as described above for the dental sensor 101 without a suction device. The head 121 is formed, for example, by a plastic insert.

Furthermore, the dental sensor 101 can also be arranged in the replaceable head 121 or along the suction tube 119. The dental sensor 101 is connected to the readout electronics 123 and can be replaced. All other components of the body-worn device 100, such as the readout electronics 123, the carrier element 105, or the holding element 103, can also be completely replaced if necessary.

The saliva is suctioned using a suction device such as a pump 125. The suction device ensures that the dental sensor 101 is moistened with saliva and that the saliva on the surface of the dental sensor 101 is replaced. The pump 125 can be used to regulate the amount of saliva or to extract the saliva at specified intervals. The extracted saliva is fed into a collection container 127 or disposed of directly in the drain.

If the dental sensor 101 is placed outside the mouth, the dental sensor 101 can also be replaced only for selective measurements by plugging it in and out. This allows a dental sensor 101 to be used that is not suitable for continuous measurements. By replacing the dental sensor 101 externally, the person 107 is not disturbed in their sporting activity during a performance test.

The body-worn device 100 may also be equipped with additional devices, such as devices for administering chemicals or medications that stimulate internal salivation. Suitable chemicals include, for example, citric acid or malic acid.

The body-worn device 100 is easy to use. No assistance from dental technicians or dentists is required to attach the body-worn device 100. The body-worn device 100 can be used by untrained personnel or even by a user in a private setting. This lowers the inhibition threshold for users to use the available dental sensors 101. The body-worn device 100 enables measurements to be taken in a simple manner, delivering reliable results. Furthermore, companies, clinics, and scientific studies can test newly developed dental sensors 101 on persons in a comparable manner.

FIG. 5 shows a schematic illustration of a method for wearing a dental sensor. The inventive method comprises the steps of holding S101 the dental sensor 101 in or on the mouth of a person 107 by means of a holding element 103; and supporting S102 the holding element 103 on the body of the person 107 by means of a carrier element 105.

All features explained and shown in connection with individual embodiments of the invention may be provided in different combinations in the subject-matter according to the invention in order to simultaneously realize their advantageous effects.

All method steps can be implemented by devices suitable for performing the respective method step. All functions performed by features of the subject-matter can be a method step of a method.

The scope of protection of the present invention is given by the claims and is not limited by the features explained in the description or shown in the figures.

### List of Reference Signs

100 Body-worn device
101 Dental sensor
103 Holding element
105 Carrier element
107 Person
109 Ergometer
111 Lactate values
113 Heart rate values
115 Fixing device
117 Interface
119 Suction tube
121 Replaceable head
123 Readout electronics
125 Pump
127 Collection container

1. A body-worn device (100) for a dental sensor (101), comprising:
a holding element (103) for holding the dental sensor (101) in or on the mouth of a person (107); and
a carrier element (105) for supporting the holding element (103) on the body of the person (107).

2. The body-worn device (100) according to claim 1, wherein the body-worn device (100) comprises the dental sensor (101).

3. The body-worn device (100) according to claim 2, wherein the dental sensor (101) is arranged at a tip of the holding element (103).

4. The body-worn device (100) according to claim 2, wherein the dental sensor (101) is replaceably attached to the holding element (103).

5. The body-worn device (100) according to claim 2, wherein the dental sensor (107) comprises an electrochemical or enzymatic dental sensor or a pH sensor, a lactate sensor, a glucose sensor, an alcohol sensor, an O2 sensor, a CO2 sensor, a CO sensor, a VO2Max sensor, a respiratory rate sensor, a noise sensor, a temperature sensor, a pressure sensor, a conductivity sensor, a humidity sensor, a potassium sensor, a sodium sensor, a chloride sensor, a cortisol sensor, a calcium sensor, a sulfate sensor, a microphone, and/or a VOC sensor.

6. The body-worn device (100) according to claim 1, wherein the holding element (103) is formed by a wire or a plastic rod.

7. The body-worn device (100) according to claim 1, wherein the holding element (103) is flexible.

8. The body-worn device (100) according to claim 1, wherein the holding element (103) comprises a fluid tube (119) for a dental sensor (101).

9. The body-worn device (100) according to claim 8, wherein the fluid tube (119) comprises a replaceable headpiece (121).

10. The body-worn device (100) according to claim 9, wherein the headpiece (121) comprises openings through which saliva can be suctioned.

11. The body-worn device (100) according to claim 1, wherein the carrier element (105) is formed by a headset, glasses, or headgear.

12. The body-worn device (100) according to claim 1, wherein the holding element (103) and the carrier element (105) are detachably connected to each other.

13. The body-worn device (100) according to claim 1, wherein the body-worn device (100) comprises an energy storage device for supplying the dental sensor with electrical energy.

14. The body-worn device (100) according to claim 1, wherein the carrier element (105) can be attached to the head or body of the person (107).

15. A method for wearing a dental sensor (101), comprising the steps of:
holding (S101) the dental sensor (101) in or on the mouth of a person (107) by a holding element (103); and
supporting (S102) the holding element (103) on the body of the person (107) by a carrier element (105).
